(19) United States
(12) Patent Application Publication
(10) Pub. No.: US 2005/0266082 A1
(43) Pub. Date: Dec. 1, 2005

(54) Preparation of stable paroxetine HC1 ER tablets using a melt granulation process

(76) Inventors: Satishkumar Ambalal Patel, West Windsor, NJ (US); Pablo Davila, East Windsor, NJ (US); Suresh Palaniswamy, East Windsor, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/129,451

(22) Filed: May 13, 2005

Related U.S. Application Data
(60) Provisional application No. 60/576,415, filed on May 26, 2004.

(51) Int. Cl. A61K 31/137; A61K 9/14; A61K 9/16

(52) U.S. Cl. 424/484; 514/649; 424/488

(57) ABSTRACT

A pharmaceutical composition comprising a hydrophobic matrix comprised of paroxetine HCl and a lipid component is provided. The matrix also preferably contains hydrophilic polymers, e.g., hypromellose. This invention also relates to a method of making such a composition by melt granulating paroxetine HCl with a molten binder comprising a lipid component. This invention also relates to tablets which contain such a matrix as a core and which having an enteric coating surrounding said core.

This application claims benefit of U.S. Provisional Application No. 60/576,415, filed May 26, 2004, which in its entirety is herein incorporated by reference.

### FIELD OF THE INVENTION

This invention relates to pharmaceutical compositions, particularly matrices which contain a pharmaceutically active ingredient and to methods of making and using same. This invention also relates to pharmaceutical tablets.

### BACKGROUND OF THE INVENTION

U.S. Pat. No. 4,839,177 discloses a system for the controlled-rate release of active substances. The system has a deposit-core comprising the active substance and having defined geometric form and a support-platform applied to said deposit-core. The deposit-core contains a polymeric material having a high degree of swelling on contact with water or aqueous liquids, which material is mixed with the active substance. The intensity and duration of the swelling force constitute the primary factor in controlling the release of the active substance.

U.S. Pat. No. 5,422,123 discloses tablets with zero order controlled-rate release of the active substance. The tablets consist of a core of defined geometric form containing the active substance and a polymer which swells on contact with aqueous liquids and a support applied to said core. The support partly covers the surface of the core and consists of polymer substances which are slowly soluble and/or gellable in aqueous liquids.

U.S. Pat. No. 6,113,944 discloses a novel pharmaceutical composition containing paroxetine. The formulation process for such composition is one in which water is absent. The process comprises dry admixing of paroxetine with excipients, compressing the mixture into a slug material or roller compacting the material into a strand material, milling the thus prepared material into a free-flowing mixture and compressing the mixture into tablets.

Despite the above process and compositions, a need still exists for paroxetine hydrochloride (HCl) in a controlled-release formulation made by a process marked by simplicity and ease of use.

### SUMMARY OF THE INVENTION

This invention relates to a pharmaceutical composition comprising a hydrophobic matrix comprised of paroxetine hydrochloride (HCl) and a lipid component. The matrix also preferably contains hydrophilic polymers. This invention also relates to a method of making such a composition by melt granulating paroxetine HCl with a molten binder comprising a lipid component. This invention also relates to tablets which contain such a matrix as a core and which have an enteric coating surrounding said core.

### BRIEF DESCRIPTION OF THE DRAWINGS

FIG. 1 and FIG. 2 each show the results of a crossover bioavailability study in vivo with the results stated as mean plasma concentration of paroxetine over time for a product of the invention and for a reference product.

### DETAILED DESCRIPTION OF THE INVENTION

Paroxetine HCl is a pharmaceutically active substance known for its utility as an anti-depressant. Paroxetine HCl can be prepared in the hemi-hydrate form as described in U.S. Pat. No. 4,721,723. Paroxetine may be prepared as described in U.S. Pat. Nos. 4,009,196, 4,902,801, 4,861,893 and 5,039,803. The amount of the paroxetine HCl in the matrix can vary broadly and will typically be from about 5% to about 25%, more typically from about 10% to 20% by weight of the matrix.

The lipid component is comprised of one or more pharmaceutically acceptable, water-insoluble materials. Preferably, the water-insoluble material has a melting point from about 30° C. to about 120° C., more preferably, from about 40° C. to about 90° C. Water-insoluble materials include, but are not limited to, waxes, spermaceti, paraffin, lecithin, fatty acids and salts or glyceride esters thereof, and C₁₂ to C₂₂ aliphatic alcohols. A mixture of water-insoluble materials may also be used. Specific examples of water-insoluble materials are beeswax, carnauba wax, microcrystalline wax, stearic acid, palmitic acid, stearyl alcohol, and cetyl alcohol. Most preferably, the water-insoluble material is stearyl alcohol.

The amount of the lipid component in the matrix can vary broadly and will typically be from about 5% to about 25%, more typically from about 10% to 20% by weight of the matrix. Such an amount should be sufficient to allow the remaining ingredients of the matrix to be melt granulated.

Preferred matrices are also comprised of one or more hydrophilic polymers. These hydrophilic polymers can affect the rate of release of the paroxetine HCl in the small intestine. Examples of such polymers are hydrophilic cellulose derivatives, such as methyl cellulose, hydroxypropyl cellulose and preferably hydroxypropyl methyl cellulose (a.k.a. hypromellose). In preferred embodiments, the matrix contains two hypromellose polymers, one having a relatively high viscosity and the other having a relatively low viscosity. In particular, the hypromellose having a higher viscosity preferably has a viscosity of at least about 500 mPas, more preferably at least about 1,000 mPas, even more preferably at least about 2,000 mPas, when measured at 2% concentration in water at 20° C. and said hypromellose having a lower viscosity preferably has a viscosity of less than about 500 mPas, more preferably less than about 250 mPas, when measured at 2% concentration in water at 20° C. The amount of the hydrophilic polymers in the matrix can vary broadly and the total will typically be from about 5% to about 25%, more typically from about 5% to 20% by weight of the matrix.

It has been found that the inclusion of the higher molecular weight hydrophilic polymer has a useful effect on the dissolution rate of the resulting tablet. In particular, the inclusion of the higher molecular weight hydrophilic polymer retards the dissolution rate of the tablet. This is useful because a slower dissolution rate leads to a lower peak plasma concentration in vivo. Thus, a target peak plasma concentration can be achieved by adjusting the ratio of the higher molecular weight hydrophilic polymer to the lower molecular weight hydrophilic polymer in the hydrophobic matrix.

The matrix will typically also be comprised of traditional excipients, such as one or more fillers, glidants, lubricants and/or surfactants. Examples of fillers include lactose, powdered sugar, calcium phosphate, calcium sulfate, microcrystalline cellulose, mannitol, kaolin, sodium chloride, dry starch and sorbitol. An example of a preferred filler is lactose which is typically present in an amount of from about 30% to about 60%, more typically from about 45% to about 55% by weight of the matrix.

Examples of glidants include colloidal silicon dioxide, talc and corn starch. A preferred glidant is colloidal silicon dioxide and is typically present in an amount of from about 0.1% to about 1%, more typically from about 0.1% to about 0.5% by weight of the matrix.

Examples of lubricants include magnesium stearate, talc, stearic acid, vegetable oil, calcium stearate and zinc stearate. A preferred lubricant is magnesium stearate and is typically present in an amount of from about 0.5% to about 3%, more typically from about 1% to about 2% by weight of the matrix. Additional excipients, such as preservatives, antioxidants, dyes and other functional additives may also be present in minor amounts.

Examples of surfactants include anionic and nonionic surfactants, e.g., fatty acid monoglycerides. A preferred class of surfactants are the anionic alkyl sulfates, e.g., sodium lauryl sulfate and is preferably used at a concentration of from about 1% to about 4% by weight of the matrix, more preferably about 2% to about 3% by weight of the matrix.

The granulation process for preparing the hydrophobic matrix will typically comprise the dry blending of the paroxetine HCl with all of the excipients with the exception of the lipid component and the lubricant. The lipid component can be pre-heated to a molten state and then mixed with the dry pre-blend of the remaining matrix ingredients. Alternatively, the lipid component can be mixed with the dry pre-blend and the entire mass is then heated to melt the lipid component. In any event, the mixing should be sufficient to homogenously disperse the dry pre-blend into the molten binder. The mixture is then allowed to cool and the mixture can be ground or milled. The lubricant can be added with mixing.

The resulting hydrophobic matrix can be filled into capsules, but is preferably formed into tablets which are then coated with an enteric coating. The enteric coating should be of a type and thickness that will prevent disintegration of the tablet in the stomach but will allow disintegration in the small intestine. Examples of such a coating include those comprised of a pH dependent anionic polymer. Preferably, such a polymer will solubilize above about pH 5.5. Examples of such polymers are the methacrylic ester polymers available from the Rohm Pharma Polymers unit of Degussa as EUDRAGIT acrylic polymers. Such polymers are typically used in an amount of from about 1% to about 6% of the weight of the tablet, more typically from about 3% to about 5% of the weight of the tablet.

### EXAMPLES

### Example 1

A heat-jacketed high shear mixer is charged with paroxetine HCl, a hypromellose polymer (HPMC) having a viscosity of about 100 mPas when measured at 2% in water at 20° C., lactose and colloidal silicon dioxide in the amounts shown in Table 1. These ingredients are mixed at 50 rpm for 15 minutes to form a dry pre-blend which is then heated to a jacket temperature of about 80° C. The amount of stearyl alcohol shown in Table 1 is then added with continued mixing and heating to about 80° C. The mixture is heated to an internal temperature of no less than 63° C. and is mixed until homogeneous. The mixture is then cooled and milled in a Fitz-mill equipped with 0.050″ screen at medium speed. Then the amount of magnesium stearate shown in Table 1 is added with continued mixing. The mixture is then tabletted with a rotary tablet B Head press equipped with 11/32″ embossed tooling. The resulting tablet cores are then coated in a perforated pan coating apparatus with a seal coat of Opadry White YS-1-7003 and water in the amounts shown in Table 1. The seal coating is followed by enteric coating in the same apparatus with the amounts of EUDRAGIT L30 D-55, triethyl citrate, talc and water shown in Table 1.

TABLE 1

| | | Quantity per Tablet |
|---|---|---|
| Ingredient | % Composition | (mg) |
| Core Tablet | | |
| Paroxetine HCl | 15.87 | 41.64 |
| Lactose Monohydrate, NF | 52.96 | 138.86 |
| Colloidal Silicon Dioxide, NF | 0.10 | 0.50 |
| Stearyl Alcohol, NF | 17.16 | 45.00 |
| HPMC K 100 LV, USP | 7.63 | 20.00 |
| Magnesium Stearate, NF | 0.10 | 4.00 |
| Seal Coating | | |
| Opadry White YS-1-7003 | 1.53 | 3.75 |
| Purified Water, USP | none | q.s. |
| Funcational Coating | | |
| Eudragit L30D 55, NF | 2.15 | 5.625 |
| Lomicron Talc, USP | 0.32 | 2.00 |
| Triethyl Citrate, NF | 0.76 | 0.85 |
| Purified Water, USP | none | q.s. |
| Total Weight | 100 | 262.225 |

Tablets were prepared substantially as described above. The results of an in vitro dissolution study of both the finished tablets and the tablet cores, are shown in Table 1A.

TABLE 1A
Dissolution Profile

| Time (Hours) | Core Tablet | Enteric-Coated Tablets |
|---|---|---|
| 0 | 0.0 | 0.0 |
| 0.5 | 32.6 | 0.1 |
| 1.0 | 48.7 | 0.2 |
| 1.5 | 71.5 | 0.2 |
| 2.0 | 83.7 | 0.2 |
| 2.5 | 88.5 | 1.0 |
| 3.0 | 93.3 | 8.5 |
| 3.5 | 96.7 | 23.5 |
| 4.0 | 98.6 | 35.9 |
| 4.5 | | 47.3 |
| 5.0 | | 54.9 |
| 5.5 | | 62.7 |
| 6.0 | | 71.9 |
| 8.0 | | 91.5 |

### Example 2

A heat-jacketed planetary mixer is charged with paroxetine HCl, a hypromellose polymer (HPMC K 4 M) having a viscosity of about 4,000 mPas when measured at 2% in water at 20° C., a hypromellose polymer (HPMC K 100 LV) having a viscosity of about 100 mPas when measured at 2% in water at 20° C., lactose and colloidal silicon dioxide, in the amounts shown in Table 2. These ingredients are mixed at 50 rpm for 15 minutes to form a dry pre-blend which is then heated to a jacket temperature of about 80° C. The amount of stearyl alcohol shown in Table 2 is then added with continued mixing and heating to about 80° C. The mixture is heated to an internal temperature of no less than 63° C. and is mixed until homogeneous. The mixture is then cooled and milled in a Fitz-mill equipped with 0.050″ screen at medium speed. Additional hypromellose as shown in Table 2 (Ingredient 6) is then added and mixed with the granules and then the amount of magnesium stearate shown in Table 2 is added with continued mixing. The mixture is then tabletted with a rotary tablet B Head press equipped with 11/32″ embossed tooling. The resulting tablet cores are then coated in a perforated pan coating apparatus with a seal coat of Opadry White YS-1-7003 and water in the amounts shown in Table 2. The seal coating is followed by enteric coating in the same apparatus with the amounts of EUDRAGIT L30 D-55, triethyl citrate, talc and water shown in Table 2.

TABLE 2

| | | | |
|---|---|---|---|
| | | | Quantity per |
| Item No. | Ingredient | % Composition | Tablet (mg) |

Core Tablet

| | | | |
|---|---|---|---|
| 1 | Paroxetine HCl | 15.16 | 42.67 |
| 2 | HPMC K 4 M, USP | 7.39 | 20.00 |
| 3 | Lactose Monohydrate, NF | 43.53 | 117.83 |
| 4 | Colloidal Silicon Dioxide, NF | 0.18 | 0.50 |
| 5 | Stearyl Alcohol, NF | 16.62 | 45.00 |
| 6 | HPMC K 100 LV, USP | 7.39 | 20.00 |
| 7 | Magnesium Stearate, NF | 1.48 | 4.00 |

Seal Coating

| | | | |
|---|---|---|---|
| 8 | Opadry White YS-1-7003 | 1.39 | 3.75 |
| 9 | Purified Water, USP | none | q.s. |
| 10 | Eudragit L30 D 55, NF | 4.16 | 11.25 |
| 11 | Triethyl Citrate, NF | 0.63 | 1.7 |
| 12 | Lomicron Talc, USP | 1.47 | 4.0 |
| 13 | Purified Water, USP | none | q.s. |
| | Total | 100 | 270.7 |

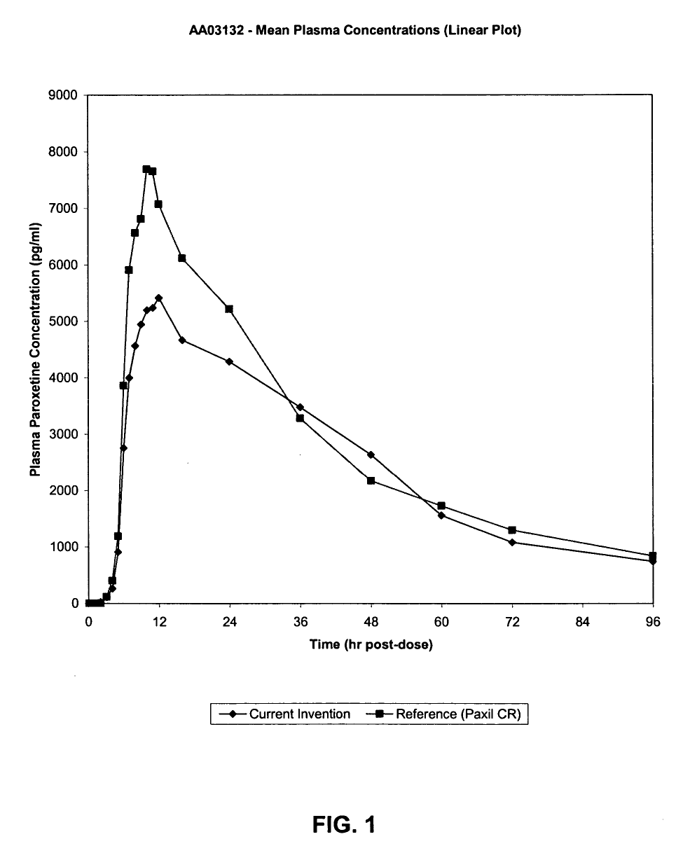

Tablets were prepared substantially as described above. The results of an in vitro dissolution study of the finished tablets are shown in Table 2A. Mean plasma concentrations from an in vivo crossover bioavailability study against the reference product from GlazoSmithKline are shown in FIG. 1.

TABLE 2A
Dissolution Result

| Time (Hours) | Average | Low | High |
|---|---|---|---|
| 0 | 0 | 0 | 0 |
| 0.5 | 0.5 | 0.4 | 0.5 |
| 1 | 0.8 | 0.7 | 0.9 |
| 1.5 | 1.0 | 1.0 | 1.1 |
| 2 | 1.0 | 0.9 | 1.1 |
| 2.5 | 4.6 | 4.3 | 5.1 |
| 3 | 8.5 | 7.5 | 9.9 |
| 3.5 | 14.4 | 13.3 | 15.9 |
| 4 | 20.8 | 19.8 | 22 |
| 4.5 | 28.7 | 27.4 | 29.5 |
| 5 | 32.4 | 31.9 | 33.2 |
| 5.5 | 38.5 | 38.2 | 39 |
| 6 | 43.9 | 43.3 | 44.5 |
| 6.5 | 50.9 | 50.1 | 51.3 |
| 7 | 54.1 | 53.3 | 54.8 |
| 7.5 | 58.6 | 57.7 | 59.7 |
| 8 | 63.6 | 62.8 | 64.5 |
| 12 | 94.7 | 92.9 | 97.0 |

### Example 3

A heat-jacketed planetary mixer is charged with paroxetine HCl, a hypromellose polymer (HPMC K 100 LV) having a viscosity of about 100 mPas when measured at 2% in water at 2020 C., lactose, colloidal silicon dioxide and sodium lauryl sulfate in the amounts shown in Table 3. These ingredients are mixed at 50 rpm for 15 minutes to form a dry pre-blend which is then heated to a jacket temperature of about 80° C. The amount of stearyl alcohol shown in Table 3 is then added with continued mixing and heating to about 80° C. The mixtures is heated to an internal temperature of no less than 63° C. and is mixed until homogeneous. The mixture is then cooled and milled in a Fitz-mill equipped with 0.050″ screen at medium speed. Then the amount of magnesium stearate shown in Table 3 is added with continued mixing. The mixture is then tabletted with a rotary tablet B Head press equipped with 11/32″ embossed tooling. The resulting tablet cores are then coated in a perforated pan coating apparatus with a seal coat of Opadry White YS-1-7003 and water in the amounts shown in Table 3. The seal coating is followed by enteric coating in the same apparatus with the amounts of EUDRAGIT L30 D-55, triethyl citrate, talc and water shown in Table 3.

TABLE 3

| | | | |
|---|---|---|---|
| | | | Quantity per |
| Item No. | Ingredient | % Composition | Tablet (mg) |

Core Tablet

| | | | |
|---|---|---|---|
| 1 | Paroxetine HCl | 16.54 | 42.67 |
| 2 | Lactose Monohydrate, NF | 50.52 | 130.33 |
| 3 | HPMC K 100 LV, USP | 7.75 | 20.00 |
| 4 | Colloidal Silicon Dioxide, NF | 0.20 | 0.5 |
| 5 | Sodium Lauryl Sulphate, NF | 2.91 | 7.5 |
| 6 | Stearyl Alcohol, NF | 17.45 | 45.00 |
| 7 | Magnesium Stearate, NF | 1.55 | 4.00 |

Seal and Functional Coating

| | | | |
|---|---|---|---|
| 8 | Opadry White YS-1-7003 | 1.45 | 3.75 |
| 9 | Purified Water, USP | none | q.s. |
| 10 | Eudragit L30 D 55, NF | 0.98 | 2.54 |
| 11 | Triethyl Citrate, NF | 0.15 | 0.38 |
| 12 | Lomicron Talc, USP | 0.50 | 1.28 |
| 13 | Purified Water, USP | none | q.s. |
| | Total | 100 | 257.95 |

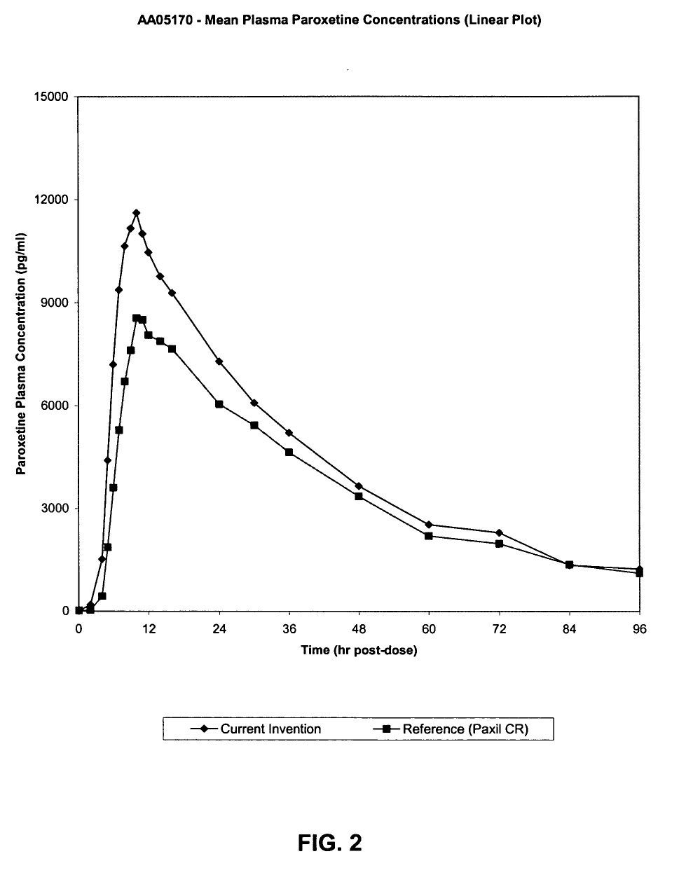

Tablets were prepared substantially as described above. The results of an in vitro dissolution study of the finished tablets are shown in Table 3A. Mean plasma concentrations from an in vivo crossover bioavailability study against the reference product from GlaxoSmithKline are shown in FIG. 2.

TABLE 3A
Dissolution Result

| Time (Hours) | Average | Low | High |
|---|---|---|---|
| 0 | 0 | 0 | 0 |
| 0.5 | 0.1 | 0 | 0.3 |
| 1 | 0.5 | 0.1 | 1.0 |
| 1.5 | 0.8 | 0.2 | 1.6 |
| 2 | 0.9 | 0.2 | 1.7 |
| 2.5 | 13.4 | 6.2 | 21.7 |
| 3 | 30.3 | 23.9 | 35.7 |
| 3.5 | 41.0 | 35.6 | 45.5 |
| 4 | 49.9 | 45.1 | 53.2 |
| 4.5 | 64.0 | 59.7 | 67.4 |
| 5 | 66.4 | 64.6 | 68.7 |
| 5.5 | 72.0 | 69.8 | 75.2 |
| 6 | 77.1 | 75.0 | 80.5 |
| 6.5 | 85.9 | 84 | 88.1 |
| 7 | 86.0 | 84.2 | 88.0 |
| 7.5 | 87.3 | 85.6 | 88.8 |
| 8 | 88.0 | 86.0 | 89.7 |
| 12 | 91.7 | 89.6 | 93.4 |

1. A pharmaceutical composition comprising a hydrophobic matrix comprised of paroxetine hydrochloride and a lipid component.

2. The pharmaceutical composition of claim 1, wherein the lipid component comprises a water-insoluble material having a melting point from about 30° C. to about 120° C.

3. The pharmaceutical composition of claim 2, wherein the water-insoluble material has a melting point from about 40° C. to about 90° C.

4. The pharmaceutical composition of claim 1, wherein the water-insoluble material is selected from the group consisting of waxes, spermaceti, paraffin, lecithin, fatty acids and salts or glyceride esters thereof, C₁₂ to C₂₂ aliphatic alcohols, and mixtures thereof.

5. The pharmaceutical composition of claim 4, wherein the water-insoluble material is selected from the group consisting of beeswax, carnauba wax, microcrystalline wax, stearic acid, palmitic acid, stearyl alcohol, and cetyl alcohol.

6. The pharmaceutical composition of claim 5, wherein the water-insoluble material is stearyl alcohol.

7. The pharmaceutical composition of claim 1, wherein the hydrophobic matrix additionally comprises one or more hydrophilic polymer.

8. The pharmaceutical composition of claim 7, wherein the hydrophilic polymer is hydroxypropyl methyl cellulose having a viscosity of at least about 500 mPas when measured at 2% concentration in water at 20° C., and hydroxypropyl methyl cellulose having a viscosity of less than about 500 mPas when measured at 2% concentration in water at 20° C.

9. The pharmaceutical composition of claim 8, wherein the paroxetine HCl is present in an amount of from about 5% to about 25% by weight of the matrix, the amount of the lipid component is from about 10% to about 20% by weight of the matrix, and the amount of the hydroxypropyl methyl cellulose polymer is from about 5% to about 25% by weight of the matrix.

10. A method of making a pharmaceutical composition comprising melt granulating paroxetine HCl with a molten binder comprising a lipid component.
